SEQUENCE LISTING (13 sequences)

SEQ ID NO: 1 - DNA, 26 bp, Homo sapiens
ttacaattgatcaccggcgaaggagg

SEQ ID NO: 2 - DNA, 25 bp, Homo sapiens
ttaatcgatgtatcttatcatgtcg

SEQ ID NO: 3 - DNA, 32 bp, Homo sapiens
accgccatggcacctacttcaagttctacaaa

SEQ ID NO: 4 - DNA, 25 bp, Homo sapiens
ttaatcgatgtatcttatcatgtcg

SEQ ID NO: 5 - DNA, 26 bp, Homo sapiens
  ref <301>: AUTHORS: Kyriakos V. Konstantinidis et al.
  ref <302>: TITLE: Targeting IL-2 to the endoplasmic reticulum confines autocrine growth stimulation to NK-92 cells
  ref <303>: JOURNAL: Experimental Hematology
  ref <304>: VOLUME: 33
  ref <305>: ISSUE: 11
  ref <306>: PAGES: 159-164
  ref <307>: DATE: 2004-11-03
  ref <308>: DATABASE ACCESSION NUMBER: 0301-472X/05
  ref <309>: DATABASE ENTRY DATE: 2004-11-03
ttacaattgatcaccggcgaaggagg

SEQ ID NO: 6 - DNA, 47 bp, Homo sapiens
tcacagttcgtccttctcgctgccagtcagtgttgagatgatgcttt

SEQ ID NO: 7 - DNA, 53 bp, Artificial Sequence
  Synthetic sequence
ggccgaattcgcggccgcaggatcctcgcgaaagcttgtcgaccaattgagct

SEQ ID NO: 8 - DNA, 525 bp, Homo sapiens
tacaattgatcaccggcgaaggagggccaccatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgagctagctcgacatgataagatacatcgattaa

SEQ ID NO: 9 - DNA, 443 bp, Artificial Sequence
  Synthetic sequence
accgccatggcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgagctagctcgacatgataagatacatcgattaa

SEQ ID NO: 10 - DNA, 515 bp, Artificial Sequence
  Synthetic sequence
ttacaattgatcaccggcgaaggagggccaccatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggcagcgagaaggacgaactgtga

SEQ ID NO: 11 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 12 - protein, 134 aa, Artificial Sequence
  Synthetic
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 13 - protein, 160 aa, Artificial Sequence
  Synthetic
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGSEKDEL

(19) United States
(12) Patent Application Publication
(10) Pub. No.: US 2006/0029583 A1
(43) Pub. Date: Feb. 9, 2006

(54) Method for the genetic activation of cells and uses of said cells

(76) Inventors: Sirac Dilber, Huddinge (SE); Alar Aints, Tartu (EE); Kyriakos Konstantinidis, Huddinge (SE)

Correspondence Address: YOUNG & THOMPSON, 745 SOUTH 23RD STREET, 2ND FLOOR, ARLINGTON, VA, 22202, US

(21) Appl. No.: 11/171,397

(22) Filed: Jul. 1, 2005

Related U.S. Application Data
(60) Provisional application No. 60/584,859, filed on Jul. 2, 2004.

(51) Int. Cl. A61K 48/00; C12N 5/08; C12N 15/867

(52) U.S. Cl. 424/93.21; 435/456; 435/372

(57) ABSTRACT

Mammalian cells, normally dependent of IL-2, can be successfully transfected to express IL-2 in amounts sufficient to sustain growth without the external addition of IL-2. One cell line expressing IL-2 solely in the endoplasmatic reticulum without secretion, and one cell line capable of secretion of IL-2 have been developed and tested. Preliminary experiments using primary cells from human donors confirm the feasibility of the invention. The invention makes available gene-modified cells, methods for their production, as well as methods of the treatment of cancer and for immunostimulation.

The present invention relates to genetic engineering in general, the production of gene modified cells, and their use in immunostimulation and in cancer therapy in particular.

### BACKGROUND OF THE INVENTION

Natural killer (NK) cells are cytotoxic lymphocytes of the innate immune system, clearly distinguishable from T and B lymphocytes [1]. They play an important role in innate immune reactions to many pathogenic microorganisms [2] [3]. In addition, they mediate strong anti-tumour responses as demonstrated in several experimental models in vitro [4] and in vivo [5]. In vivo, they can control growth and metastatic spread [6]. NK cells may also contribute to the resistance to human malignancies, clearly demonstrated in settings of stem cell transplantation for the treatment of haematological malignancies. These observations have prompted several studies aimed at enhancing human NK cell activity in vivo in cancer patients, e.g. by using specific cytokines or other stimuli to directly enhance NK cell activity in vivo [7].

Anti-tumour effects mediated by NK cells can be enhanced by cytokines, including interleukin 2 (IL-2), IL-12, IL-15, IL-18 and IL-21 [8-11] [12]. A number of attempts have been made to administer IL-2 systemically to cancer patients. These strategies have been met with mixed clinical results dependent on protocol, type and stage of cancer, and other factors. Systemic IL-2 administration is however frequently associated by undesirable side effects [13-15], such as toxicity affecting the cardiovascular, gastrointestinal, respiratory and nervous systems. The latter includes difficulties in thinking, mood changes, loss of appetite and flu-like symptoms. In settings where IL-2 is given primarily to enhance NK activity, administration in a form that would stimulate NK cells yet not give any unwanted side effects would be ideal. This has prompted investigations for alternative approaches for IL-2 delivery.

NK-92 is an in vitro propagated NK cell line with phenotypic and functional characteristics of primary human NK cells [16]. This cell line is strictly dependent on IL-2 for its growth and survival. This makes it a particularly useful model for studies of IL-2 effects on NK stimulation. Several attempts have been made to transduce or by other means introduce functional IL-2 genes to NK-92 cells. These studies have shown that IL-2 expressed by these means fulfil the purpose of stimulating NK-92 growth and survival.

The main aim underlying the present invention was to find alternatives to a systemic IL-2 administration, as well as to eliminate the necessity of adding IL-2 for ex vivo culture of IL-2 dependent cells, such as T-cells and NK cells. One particular aim of the present-inventors was to generate auto activated NK cells. Other aims and objectives, and the solutions offered by the invention, as well as the advantages associated therewith, will become evident to a skilled person from the study of the description, non-limiting examples and claims.

### SHORT SUMMARY OF THE INVENTION

The present inventors have surprisingly found that mammalian cells, dependent on IL-2, can be successfully transduced to express IL-2, and in particular a non-secreted form of IL-2, in amounts sufficient to sustain growth without the external addition of IL-2. In the present study, the present inventors have cloned and expressed three different forms of IL-2 in cultured mammalian cells. Expression of the constructs was verified by immunostaining of transduced Cos-7 cells. Biological activity of the modified proteins was assessed in NK-92 cells. Surprisingly, an endoplasmic reticulum (ER) retained form of IL-2 sufficed to promote growth and survival of NK-92 cells. Furthermore, such cells expressed retained cytotoxic potential. This study demonstrates that it is possible to express IL-2 in NK-92 cells in a way that prevents secretion of the cytokine and thus any possible unwanted side effects. The implications of the present findings are discussed.

The invention makes available gene-modified cells expressing IL-2, methods for their production, as well as methods of treatment, such as immunostimulation or immunotherapy, as defined in the attached claims, incorporated herein by reference.

The invention is exemplified by the examples, showing that a genetically modified NK-92 cell line, NK92IL2ER, express IL-2 in a restricted area of the endoplasmic reticulum at levels sufficient for NK cell survival. Another genetically modified NK-92 cell line, NK92IL2WT, is capable of secreting IL-2 in amounts comparable to that of non-modified activated T-cells. Preliminary experiments performed during the priority year indicate that the results are transferable to human primary cells from healthy donors.

### BRIEF DESCRIPTION OF THE DRAWINGS

The invention will be described in closer detail in the following description, examples, and attached drawings, in which

FIG. 1 shows a schematic representation of plasmids expressing modified forms of IL-2. L: leader; IL-2: interleukin 2; ERRS: endoplasmic reticulum retention signal.

FIG. 2 is a bar diagram, showing the proliferation of IL-2 transduced and parental NK-92 cells in a short-term six-day culture. NK-92 cells were cultured with an initial concentration of 25000 cells/ml in a 90% CellGro®, 10% FBS and 500 IU/ml IL-2. At day 0, the cells were washed thoroughly with PBS to remove any traces of IL-2 from previous culturing stages. Non-modified NK-92 cells die in the absence on IL-2, while gene-modified cells proliferate comparably to the cells in the presence of added IL-2.

FIG. 3 is a bar-diagram showing the results of co-cultivation of NK-92 GFP with NK-92 IL-2-modified expressing cells in 1:1 ratio. 25000 IL-2 expressing NK-92 cells were mixed with an equal number of NK-92 cells expressing GFP. Both cell populations were thoroughly washed twice with PBS in order to exclude any traces of external IL-2 from previous culturing. The proportion of GFP positive and negative cells was quantified after 48 hours by FACS analysis. NK-92 GFP cells die in the culture with NK-92IL2ER, which proves the absence of secretion of IL-2 from these cells.

FIG. 4 is a bar-diagram illustrating of the gene-modified NK-92 production of IL-2 in vitro. Cells were cultured for 6 days, with a starting concentration of 25000 cells/ml. Supernatants were harvested 48 hours after medium change. The levels of IL-2 in the supernatants were measured using enzyme linked immunoabsorbent assay (ELISA) as described in the Materials and Methods section. The results show that the IL-2 production and secretion of NK92IL2WT is comparable to that of T-cells, whereas the IL-2 levels in the supernatant of NK92IL2ER cells are comparable to medium only (control).

FIG. 5 shows the cytotoxicity of the parental and gene-modified cells against K-562 cells in a 4-h ⁵¹Cr release assay. The gene-modified cells retain their cytotoxic potential comparable to the non-modified cells.

### DESCRIPTION

Before the present invention is described, it is to be understood that the terminology employed herein is used for the purpose of describing particular embodiments only and is not intended to be limiting, since the scope of the present invention will be limited only by the appended claims and equivalents thereof.

The terms “treatment”, “therapy”, “therapeutic use”, “medicament”, and “medical use” encompass both human and animal or veterinary applications.

The term “functionally equivalent” defines a protein or nucleotide sequence, having a different amino acid or base sequence, compared to the sequences disclosed herein, but exhibiting the same function in vitro and in vivo. An example of a functional equivalent is a modified or synthetic gene, encoding the expression of a protein identical or highly homologous to that encoded by the wildtype gene.

“Transfection” means gene transfer by physical or chemical means. “Transduction” means gene transfer by viral vectors. “Stable genetic modification” implies inheritability of the genetic modification, usually (but not necessarily) achieved by integration of the transferred DNA into one of the cell's chromosomes. “Transient genetic modification” implies loss of the genetic material in the time course of weeks or months.

According to an embodiment, the present invention makes available a method of producing an IL-2 expressing mammalian cell, comprising the steps of
selection or construction of an IL-2 gene,
preparation of a retroviral vector carrying said IL-2 gene,
collection of cells from a donor,
genetic modification of said cells, and
optionally, selection of transfected cells.

Said IL-2 gene is preferably a gene encoding the protein of SEQ. ID. NO. 11 or functional equivalents thereof. Said IL-2 gene is more preferably a gene encoding the protein of SEQ. ID. NO. 13 or functional equivalents thereof. Most preferably, said gene is chosen among SEQ. ID. NO. 8, SEQ. ID. NO. 10, or functional equivalents thereof.

According to one embodiment, the mammalian cells are chosen among natural killer (NK) cells, and T-cells, preferably natural killer (NK) cells.

According to another embodiment, said modified IL-2 gene is modified to direct the expression of IL-2 to the endoplasmatic reticulum of said cell.

The invention further makes available a transgenic mammalian cell capable of producing IL-2, produced by the method outlined above and specified in closer detail in the examples.

The invention concerns in particular mammalian cells, which in their non-transfected state are dependent of IL-2 for their growth, and unable to produce any significant amounts thereof, wherein said transgenic cells produce IL-2 in an amount sufficient to sustain growth without the need of external IL-2.

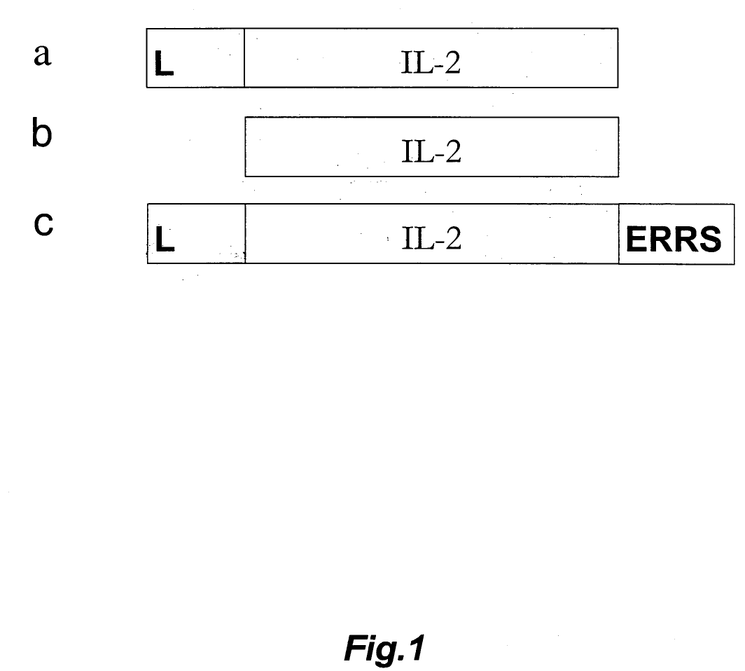

According to a preferred embodiment, IL-2 expression is restricted to the endoplasmatic reticulum of said cells. It is important to note, that simply preventing IL-2 secretion, for example, by removing the secretion signal, (construct 2, FIG. 1) is not sufficient to provide autocrine growth stimulation to gene-modified cells.

The invention also makes available methods of therapy, including palliative, curative and prophylactic treatment. According to one embodiment, the invention relates to a method in the treatment of cancer, comprising the administration to said patients a therapeutically efficient and physiologically acceptable amount of transgenic cells as defined above. The cancer can be any form of cancer, including but not limited to cancer of the colon, prostate, breast, kidneys, liver, stomach, lungs, brain, and skin (melanoma), including leukaemia and lymphoma. Presently, the effects of IL-2 administration have been shown mainly on metastatic renal cancer and metastatic melanoma.

The invention also makes available a method of immunostimulation using gene modified cells, expressing substantially physiological levels of IL-2, wherein an IL-2 expressing mammalian cell is produced through a method, comprising the steps of: selection or construction of an IL-2 gene; preparation of a retroviral vector carrying said IL-2 gene; collection of cells from a donor or patient; genetic modification of said cells; optionally, selection of gene-modified cells, and administration of said modified cells to a patient in need thereof.

As above, said IL-2 gene is preferably a gene encoding the protein of SEQ. ID. NO. 11 or functional equivalents thereof. Said IL-2 gene is more preferably a gene encoding the protein of SEQ. ID. NO. 13 or functional equivalents thereof. Most preferably, said gene is chosen among SEQ. ID. NO. 8, SEQ. ID. NO. 10, or functional equivalents thereof.

The invention also makes available a method for stimulating the immune system of a patient, comprising the administration to said patients a therapeutically efficient and physiologically acceptable amount of transgenic cells as defined above. It is contemplated that said immunostimulation or immunotherapy would constitute a step in the treatment of cancer, or treatment or prevention of an infection. As defined above, the cancer can be any form of cancer, including but not limited to cancer of the colon, prostate, breast, kidneys, liver, stomach, lungs, brain, and skin (melanoma), including leukaemia and lymphoma

In the above methods, said cells are preferably taken from the patient, gene-modified, and returned to said same patient. However, it is also contemplated that said cells are taken from a donor, gene-modified, and administered to said patient.

Said use of transgenic cells may constitute an adjunct or supplementary therapy, performed before, after or substantially simultaneously with another therapy. In the treatment of cancer, said transgenic cells can be administered before, after or substantially simultaneously with cytotoxic drugs, radiation therapy, surgical intervention, or a combination thereof. It is however also contemplated that said use of transgenic cells would constitute the primary therapy in the treatment of cancer.

The invention also encompasses the use of said cells for the manufacture of pharmaceutical compositions or medicaments, for use in the treatment of cancer, or treatment or prevention of an infection. The type of cancer may be any type of cancer, but is preferably one of the cancer types defined above.

The work of the present inventors demonstrates the feasibility of conferring a strictly autocrine signalling mode to a naturally systemically acting cytokine, IL-2, by adding an ER retention signal to the cytokine's coding sequence. The retrovirally gene-modified NK-92 natural killer cell lines continue to proliferate in the absence of exogenously added IL-2. Previous studies [26, 27] have reported NK-92 gene modifications by stable transfection. TR-IL-2-NK-92 cells produce 5.5 ng/10⁶/24 hours, NK-92MI produce 1260 pg/ml/48 hours/10⁶ and NK-92CI 15 pg/ml/48 hours/10⁶. Several of these cell lines produce significantly higher level of IL-2 compared to primary cells, which can be explained by multicopy plasmid integration during transfection and can be considered potentially harmful. The inventive NK-92IL2WT produces 18, 3 pg/ml/48 hours/10⁶, which is comparable to activated primary human T-cells (40 pg/ml/48 hours/10⁶).

The secretion of the ER-targeted construct was comparable to background, yet the cells proliferated well. This surprising finding can be explained by binding of the ER-retained IL-2 to its receptor in the ER en route to the cell membrane. Also, signalling from receptor-ligand complexes directly from ER is possible. Similar modes of signalling have been described for GM-CSF [29] and IL-3 [30].

Systemic administration of IL-2 to patients to support the transferred immune effector cells is accompanied by strong side effects. Delivery of NK cells capable of supporting their own proliferation as the IL2ER-cells, providing stimulation to the surrounding immune cells via cytokines such as TNF-a and IFN-g naturally produced by activated NK cells, and additional local secretion of IL-2 (IL2WT cells) without the side-effects of systemic injections can be advantageous in cancer immunotherapy for a direct antitumor effect, as well as in other applications, where immunostimulation is desired.

A transgenic IL-2 producing cell has a surprising advantage in that it is likely to become resistant to the defence mechanisms of tumour cells, usually silencing NK cells. The IL-2 production resulting from the gene transfer gives the cell auto stimulating properties.

In an embodiment, where the transgenic IL-2 producing NK cell also secretes IL-2, it will stimulate surrounding, non-transfected cells, and provide a localised stimulating effect on the immune system via secreted IL-2 at physiological levels, which has previously not been achieved with other methods.

In conclusion, the present inventors have made available gene-modified cells for cancer immune therapy, capable of direct antitumor effect in the absence of systemic support in the form of IL-2 injections, also capable of localised immune stimulation via localised secretion of cytokines naturally produced by NK cells, or additional IL-2. The inventors have shown the ability of ER-retained IL-2 to provide autocrine growth stimulation to the gene-modified cells, without secretion of the cytokine to extracellular compartment.

Another advantage of the invention is that time consuming and costly ex vivo culturing steps can be avoided. The time savings alone are considerable, but also the fact that ex vivo handling of cells that are to be administered to a patient is strictly regulated, makes this an important improvement.

Further, the administration of IL-2 in order to support cell growth, whether ex vivo or in vivo, is no longer necessary. In the in vivo application, the side effects of systemic IL-2 administration are avoided.

Other problems of the prior art, overcome by the inventive methods and modified cells, as well as the advantages associated therewith, will be evident to a skilled person upon closer study of the present description, examples, figures and attached sequence listing.

### EXAMPLES

Materials and Methods

Cell Lines

The cell line Phoenix GP was used for retrovirus production (with permission from Dr. Garry P. Nolan, Ph.D., Department of Molecular Pharmacology, Stanford University School of Medicine, Stanford, Calif.). Cos-7 (DSMZ, Braunschweig, Germany), a cell line derived from African green monkey was used for immunostaining. Both cell lines were cultured in Dulbecco's modified Eagle's medium (DMEM—Invitrogen Corporation, Paisley, Scotland) with Glutamax, sodium pyruvate, 4500 mg/l glucose and pyridoxine, supplemented with 10% fetal bovine serum (FBS—Invitrogen).

NK-92 cell line was purchased from LGC Promochem/ATCC (Boras, Sweden). NK-92 cells were maintained in stem cell medium (CellGro®) supplemented with 10% FBS and 500 IU/ml IL-2 (Proleukin®, Chiron, Calif., USA). CeliGro® SCGM is a GMP (good manufacturing practice) quality serum-free medium for culture of hematopoietic stem and progenitor cells (CellGenix, Freiburg, Germany). Proleukin® is a GMP quality interleukin-2 and was aliquoted and stored at −20° C. at 10⁶ IU/ml stock concentration.

K-562 (LGC Promochem/ATCC, Boras, Sweden), a human myeloid leukaemia cell line, was used as a target for natural killer cells. K-562 cells were cultured in RPMI 1640 medium (Invitrogen), supplemented with 10% FBS.

All cell lines were incubated at 37° C., 5% CO₂ and 95% humidity and were subcultured every 2-3 days. All culture mediums were stored at +4° C. and FBS was heat inactivated at 56° C. for 1 hour and stored at −20° C. Aliquots of cells from early passage were frozen in 10% dimethyl sulfoxide (DMSO—Sigma-Aldrich, St.Louis, Mo., USA)/90% FBS and stored at −150° C. for later reconstitution. Phosphate-Buffered Saline (PBS) without calcium, magnesium and sodium bicarbonate, was purchased from Invitrogen and stored at 4° C.

All cell populations were observed using an inverted microscope (Olympus CK40) with a UV module (Olympus U-RFLT50) at regular intervals and were monitored regularly for cell viability with trypan blue exclusion and for mycoplasma contamination. For data acquisition and analysis, a FACSCalibur was used along with Cell Quest™ 3:3 Analysis Software (Becton Dickinson, Calif., USA). In each sample, at least 10000 cells were acquired in the analysis region of viable cells, defined by side and forward scatter.

Plasmids

The pORF-hIL2 plasmid, containing the IL-2 cDNA template was purchased from InvivoGen (San Diego, Calif., USA). The required IL-2 primers were designed using Oligo 6.6 software (Molecular Biology Insights Inc, CO, USA) and they were ordered from DNA Technology ApS, Arhus, Denmark. The IL-2 variants were cloned by PCR using the following primers:

SEQ. ID. NO. 1: wild-type IL-2 [TTA CAA TTG ATC ACC GGC GAA GGA GG] (forward), and

SEQ. ID. NO. 2: [TTA ATC GAT GTA TCT TAT CAT GTC G] (reverse);

SEQ. ID. NO. 3: cytoplasmic (leaderless) IL-2 [ACC GCC ATG GCA CCT ACT TCA AGT TCT ACA AA] (forward), and

SEQ. ID. NO. 4: [TTA ATC GAT GTA TCT TAT CAT GTC G] (reverse); and

SEQ. ID. NO. 5: endoplasmic reticulum IL-2 [TTA CAA TTG ATC ACC GGC GAA GGA GG] (forward), and

SEQ. ID. NO. 6: [TCA CAG TTC GTC CTT CTC GCT GCC AGT CAG TGT TGA GAT GAT GCT TT] (reverse, including endoplasmic reticulum retention signal).

The PCR products were cloned into pCR®4BluntTOPO® vector (Invitrogen). The TOPO® cloning and transformation steps were performed according to manufacturer's instructions. Clones were analysed using restriction analysis and cycle sequencing and subcloned with EcoRI into pSF91-MCSg.

pSF91-MCSg was derived from the mouse leukaemia virus-based retroviral vector pSF91-GFP-gPRE, a kind gift from Prof. Christopher Baum (Hanover Medical School, Hanover, Germany). To facilitate further construction, the NotI-HindIII fragment (containing GFP and gPRE) was replaced by a synthetic oligonucleotide cloning site (SEQ. ID. NO. 7),

containing restriction sites for EcoRI, NotI, BamHI, HindIII, NruI, SalI and MfeI. Thereafter, gPRE element, as an EcoRI fragment was reinserted into MfeI site to make pSF91-MCSg. For eGFP vector construction, the eGFP gene from pEGFP-N3 (Clontech, Palo Alto, Calif.) was released with HindIII-NotI (filled in) and inserted into pSF91-MCSg between HindIII-SalI (filled in), and the resulting plasmid was called pSF91-GN3g. All constructs were confirmed by restriction mapping and partial sequencing. Finally three constructs were prepared; a first sequence (SEQ. ID. NO. 8) expressing wild type IL-2 (secreted),

As comparison, a second sequence (SEQ. ID. NO. 9) targeted to the cytoplasm was constructed.

Finally, a third sequence, targeted to the endoplasmic reticulum (SEQ. ID. NO. 10) was constructed.

For a schematic illustration of the plasmids, see FIG. 1

The corresponding amino acid sequences are also given in the attached sequence listing:

SEQ. ID. NO. 11: IL-2 wild type

SEQ. ID. NO. 12: Leaderless IL-2

SEQ. ID. NO. 13: IL-2 ER

Plasmid DNA was purified using QIAprep 8 Turbo Miniprep, QIAprep 8 Miniprep and Qiagen® Plasmid Maxi (Qiagen Inc., Calif., USA) and in some cases the Genelute HP Plasmid Midiprep Kit (Sigma-Aldrich). All the above kits were used according to manufacturer's instructions.

Transfection and Transduction

Phoenix GP cells and Cos-7 cells were transiently transfected with 3 μg and 2 μg of vector construct plasmid, respectively, and 1 μg PMD-G (encoding vesicular stomatitis virus envelope glycoprotein, kindly provided by Dr. D. Trono, Dept. of Genetics and Microbiology, University of Geneva, Geneva, Switzerland) per 35 mm cell culture well. The transfection of PhoenixGP leaded to collection of virus supernatant for transduction experiments and the transfected Cos-7 cells were used for immunostaining purposes. For transfections, Fugene 6 reagent (Roche Boehringer Mannheim, Germany) was used according to manufacturer's instructions. Briefly, DNA plasmid vectors and Fugene reagent were mixed at ½ mass/volume ratio in 100 μl volume of cell culture medium and added to cells after 15 minutes. For positive control, a GFP containing plasmid (pSF91-GN3g) in a retroviral backbone was used. Supernatant was collected 24 and 48 hours after transfection, filtered through 0.45 μm Millex-GP syringe-top filter (Millipore Corporation, Bedford, Mass.) and used immediately for transduction. The efficiency of transfection, in positive controls, was always higher than 50%.

The vector-containing supernatant was used to transduce NK-92 cells, and the cells were centrifuged at 1000×g for 1 hour in the presence of 300 μl of IL-2 supernatant and 4 μg/ml hexadimethridine bromide (Polybrene®, Sigma-Aldrich).

Proliferation

The biological activity of the expressed inteleukin-2 was determined by a cell proliferation assay, using the IL-2 dependent cell line NK-92. The cell growth was quantified by counting cells in a Bürcher chamber with Trypan Blue viability stain, and with the cell proliferation reagent WST-1 (Roche Boehringer Mannheim) according to manufacturer's recommendations.

Quantitative Analysis of Secreted Interleukin-2 in Supernatants of IL-2 Transduced NK-92 Cells

For the quantitative determination of human interleukin-2, the OptEIA™ Human IL-2 ELISA Kit II (BD Biosciences Pharmingen, San Diego, Calif., USA) was used according to manufacturer's instructions.

Immunostaining

IL-2 modified Cos-7 cells were washed with PBS and fixed with 4% paraformaldehyde for 15 minutes at room temperature (RT). After fixing, cells were rinsed with PBS and incubated with NP40 (Vysis Inc, Ill., USA) −1% in PBS- for 10 minutes at RT. Then cells were PBS washed three times and blocked with blocking buffer for 30 minutes, containing 0.1% Tween 20 (Sigma-Aldrich), 0.1% BSA-c (Aurion, Netherlands) and 5% goat serum (DAKO A/S, Glostrup, Denmark) in PBS. Cells were washed three times for 4 minutes with PBS/0.1% Tween 20 and incubated for 45 min with 5 μg/ml primary purified rat anti-human IL-2 antibody (BD Biosciences Pharmingen) diluted into blocking buffer. Cells were then again washed four times for 5 minutes with PBS/0.1% Tween 20 and incubated for 1 hour with 5 μg/ml secondary Oregon green 488 nm goat anti-rat IgG antibody in blocking buffer. Finally cells were rinsed with PBS/Tween 20 and counterstained with Hoechst stain (Molecular Probes BV, Leiden, Netherlands) at 1:4000 dilution in PBS for 5 minutes at room temperature followed by one PBS wash. Cells were visualised by fluorescence microscopy with a Leica DMRXA microscope (Leica Microsystems, GmbH, Wetzlar, Germany) equipped with a CCD camera (model S/N 370 KL 0565, Cooke Corporation, NY, USA). Filter sets for DAPI/Hoechst, FITC, Cy3 and Cy5 were obtained from Chroma technology (Brattleboro, Vt., USA). The images were acquired using the Slidebook 2.1.5 software (Intelligent Imaging Innovations Inc, Denver, Colo., USA) and Adobe Photoshop 5.0 (Adobe Systems, Seattle, Wash., USA)

In order to verify that the locus of IL-2 expression is indeed in the endoplasmic reticulum, a second immunostaining was performed. After transduction, NK-92 cells were stained with ER-Tracker™Blue-White DPX (Molecular Probes, Eugene, USA) according to manufacturer's instructions. ER-Tracker stains specifically the endoplasmic reticulum. After washing with PBS, cells were fixed with 2% paraformaldehyde, permeabilised with NP40 and stained with IL-2 antibodies as described above.

Co-Cultivation of NK-92 Cells Expressing Different Forms of IL-2 With GFP Modified NK-92 Cells

NK-92 cells modified to express green fluorescent protein (GFP) were mixed, into 1:1 ratio with IL-2 modified NK-92 cells. Co-cultivation experiments were carried out in six-well plates as follows: 25000 IL-2 expressing NK-92 cells were mixed with an equal number of NK-92 cells expressing GFP. Both cell populations were thoroughly washed twice with PBS in order to exclude any traces of external IL-2 from previous culturing. The proportion of GFP positive and negative cells was quantified after 48 hours by FACS analysis.

Cytotoxicity Assay

The cytotoxic function was measured in a standard 4 h ⁵¹Cr-release assay in triplicates. Briefly, 1×10⁶ K562 cells were labelled with 100 μl ⁵¹Cr and were incubated for one hour at 37° C. Effector cells were counted using trypan blue exclusion dye and mixed with target cells to obtain an effector:target ratio of 10:1, 3:1, 1:1 and 0.3:1.

CellGro medium was used as negative control and for positive control cells were incubated with 1% of Triton X. After incubating into a V-bottom shape 96-well plate for 4 hours at 37° C., 70 μl of supernatant were aspirated from each well and counted using a Packard Cobra Auto-Gamma 5000 Series Counting system (Meriden, Conn., USA). The percentage of the spontaneous release was calculated from the following formula: %51 Cr release=(sample-spontaneous)/(max release−spontaneous)×100.

During the priority year, further experiments were performed, using primary cells from healthy donors.

Primary Donor Cell Culture

Buffy-coat cells were obtained from healthy blood-bank donors at Karolinska University Hospital, Huddinge, Stockholm, Sweden and cultures were initiated on the same day (day 0). PBMCs were isolated by gradient centrifugation, using Lymphoprep (Nyegaard, Oslo, Norway). After washing twice with phosphate-buffered saline (PBS) (Gibco), cell viability was assessed by trypan blue dye exclusion, and the cells were plated onto six-well dishes (Falcon by Becton Dickinson, Le Pont de Claix, France) at 0, 25×10⁶ cells/ml. CelIGro SCGM medium (CellGenix, Freiburg, Germany) was used in all the cultures with the addition of 5% human serum (BioWhittaker, Cambrex Bio Science, Walkersville, Md., USA), 500 IU/ml Interleukin-2 (IL-2) (Peprotech, N.J., USA), and 10 ng/ml anti-CD3 antibody, OKT-3 (Ortho Biotech Inc. Raritan, N.J., USA). On day 5, OKT-3 was washed out, and cells were thereafter cultured in CelIGro medium supplemented with 500 IU/ml IL-2 and 5% human serum without OKT-3. Then, fresh medium was added to cultures every 1-2 days until day 21. Absolute cell counts (ACC) of cell subsets were obtained by multiplying percentage of cell subsets with the total cell number of the culture at the same time point.

Retroviral Transduction of NK Cells

The stably transduced retrovirus producer cell lines, producing SF91g-IL2wt, SF91g-IL2-L, SF91g-IL2ER and SF91g-GN3 (GFP control) retroviral vectors were grown in DMEM supplemented with 10% FCS. When the cells were subconfluent, new fresh medium was added for 24 hours. Finally the supernatant was collected, filtered through a 0.45 μm filter (Millipore, Billerica, Mass., USA), and frozen at −70° C. The supernatant collected and harvested had a titer of 0, 5×10⁶ virus particles per ml measured on HeLa cells. Viral particles produced from this producer cell line contain a GALV (Gibbon Ape Leukemia Virus) envelope. All transductions were carried out by replacing the media with the retrovirus containing supernatant at a multiplicity of infection (MOI) of 3, in the presence of 8 μg/ml polybrene (Sigma) and 500 U/ml IL-2 by centrifugation at 1.000 g at room temperature for two hours. After centrifugation, the supernatant was replaced with the NK-cell medium (CelIGro medium with 500 U/ml IL-2 and 5% human serum) and cells were expanded until day 21. As a control, PBMCs of the same donor were grown and mock infected under similar conditions as transduced cells.

Results

Effects on Cell Proliferation From Expression of IL-2 in NK-92 Cells

Three constructs for the expression of IL-2 were evaluated; one expressing wild type IL-2, one targeting IL-2 to the cytoplasm and one targeting IL-2 to the endoplasmic reticulum (FIG. 1). The cytoplasmic construct was biologically inactive (data not shown).

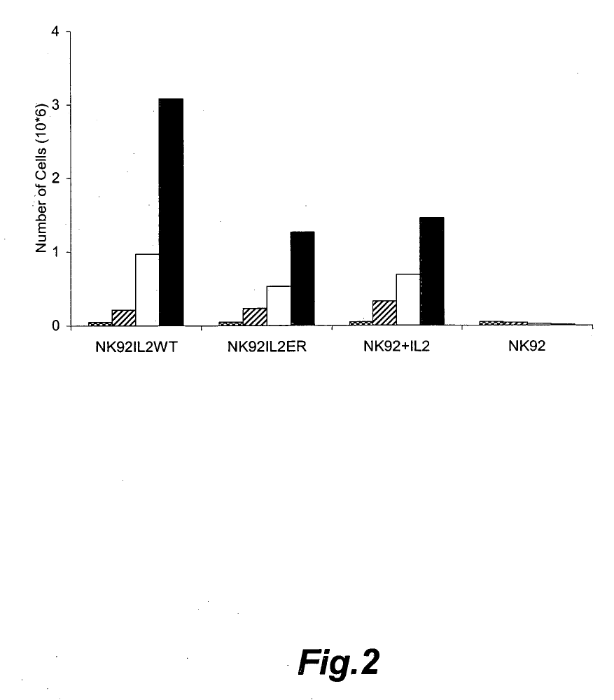

After transduction, the cells were cultured 4 days in the presence of IL-2 in order to allow for gene expression and then extensively washed in PBS and kept either with or without IL-2 supplement in the culture conditions. Two IL-2 transduced cell lines, developed by the inventors; the NK92IL2WT and the NK92IL2ER were selected for this study. As shown in FIG. 2, non-transduced NK-92 control cells died in the culture 6 days after IL-2 removal. In contrast, NK92IL2WT and NK92IL2ER exhibited a similar growth curve as NK-92 cells supplemented with external IL-2 (FIGS. 2).

The growth characteristics of NK92IL2WT and NK92IL2ER have been stable after continuous culture for almost a year.

Localised and Restricted IL-2 Expression in the Endoplasmic Reticulum

The functionality of the vector for localised expression in the endoplasmatic reticulum was first analysed through transient transfection into Cos-7 cells. The Cos-7 cells were chosen for the monitoring of transduced protein expression because of its clear morphology during microscopic imaging. Indirect immunofluorescence of IL-2ER transduced Cos-7 cells using a monoclonal rat anti-human IL-2 antibody showed bright green staining compatible with endoplasmic reticulum localisation. (Data not shown).

Expression of IL-2 in the Endoplasmatic Reticulum Does not Generate Bystander Growth Support to Neighbouring Cells

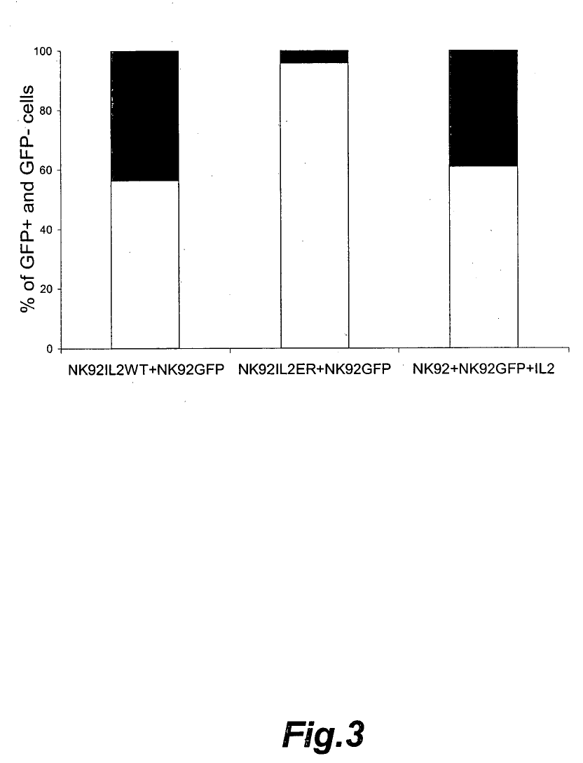

Next, the present inventors wanted to investigate whether small amounts of IL-2 leak into the surrounding culture medium from the transduced cells and if this would be sufficient for growth support of the parental NK-92 cells. For this, equal numbers of NK-92 IL-2 modified and GFP-modified NK-92 cell lines were co-cultured and evaluated for growth. After 48 hours of culturing the percentage of NK-92 GFP cells was severely reduced (45%) when mixed with NK92IL2ER. In contrast, when mixed with NK92IL2WT cells a growth support was observed (FIG. 3). After two days of culturing, the proportion of both populations still remains close to the initial 50%, indicating that NK92IL2WT secrete IL-2 to the supernatant, supporting the survival of NK92GFP.

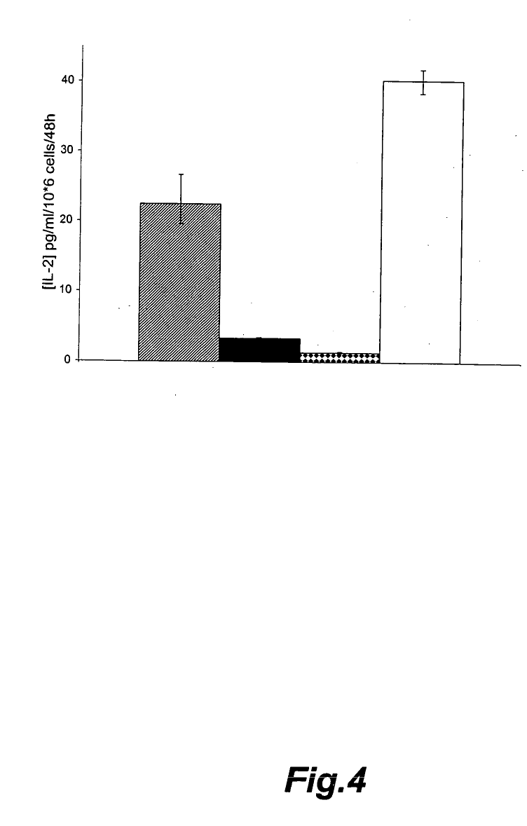

The amount of IL-2 in the supernatant was quantified by enzyme-linked immunosorption assay. The IL-2 level detected in the supernatant from NK92IL2ER cells was 3, 2 pg/ml/10⁶ cells/48 h (FIG. 4). This is comparable to background (CelIGro® medium with FCS). The IL-2 concentration in supernatants from NK92IL2WT was 18, 3 pg/ml/10⁶ cells/48 h and a control supernatant from T cells activated with anti-CD3 antibody showed 40, 3 pg/ml/10⁶ cells/48 h. Preliminary laboratory data indicates that 30 ng/ml (500 IU/ml) of IL-2 leads to optimal growth in non-modified NK-92 cells (data not shown).

The Genetically IL-2 Modified NK-92 Cell Populations Show Similar Cytotoxic Effects Compared to the Parental NK-92 Cell Line

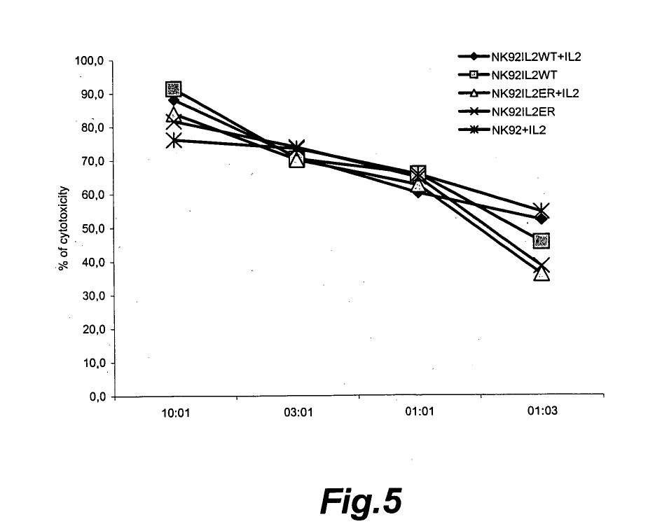

The NK92IL2WT and NK92IL2ER lines could lyse 51 Cr-labelled K562-cells at a level comparable to the parental NK-92 cells. (FIG. 5). A 1:1 effector: target ratio yielded a median cytotoxic activity of 59.2% (NK92IL2WT) and 46.4% (NK92IL2ER), compared to 59% for the NK-92 cells.

The preliminary experiments carried out during the priority year on primary NK cells expanded from donor PBMCs demonstrated the ability of the transferred IL2 wt (SEQ. ID. NO. 8) and IL2ER (SEQ. ID. NO. 10) genes to support the growth of primary human NK cells in the absence of exogenously added IL-2.

Although the invention has been described with regard to its preferred embodiments, which constitute the best mode presently known to the inventors, it should be understood that various changes and modifications as would be obvious to one having the ordinary skill in this art may be made without departing from the scope of the invention which is set forth in the claims appended hereto.

### REFERENCES

1. Trinchieri G Biology of natural killer cells. Adv Immunol. 1989; 47:187-376
2. Unanue E R Studies in listeriosis show the strong symbiosis between the innate cellular system and the T-cell response. Immunol Rev. 1997; 158:11-25
3. Biron C A, Nguyen K B, Pien G C, Cousens L P, Salazar-Mather T P Natural killer cells in antiviral defense: function and regulation by innate cytokines. Annu Rev Immunol. 1999; 17:189-220
4. Orange J S, Fassett M S, Koopman L A, Boyson J E, Strominger J L Viral evasion of natural killer cells. Nat Immunol. 2002; 3:1006-1012
5. Wu J, Lanier L L Natural killer cells and cancer. Adv Cancer Res. 2003; 90:127-156
6. Kiessling R, Klein E, Wigzell H “Natural” killer cells in the mouse. I. Cytotoxic cells with specificity for mouse Moloney leukemia cells. Specificity and distribution according to genotype. Eur J Immunol. 1975; 5:112-117
7. Seaman W E, Sleisenger M, Eriksson E, Koo G C Depletion of natural killer cells in mice by monoclonal antibody to NK-1.1. Reduction in host defense against malignancy without loss of cellular or humoral immunity. J Immunol. 1987; 138:4539-4544
8. Kim S, Iizuka K, Aguila H L, Weissman I L, Yokoyama W M In vivo natural killer cell activities revealed by natural killer cell-deficient mice. Proc Natl Acad Sci U S A. 2000; 97:2731-2736
9. Lowdell M W Natural killer cells in haematopoietic stem cell transplantation. Transfus Med. 2003; 13:399-404
10. Parham P, McQueen K L Alloreactive killer cells: hindrance and help for haematopoietic transplants. Nat Rev Immunol. 2003; 3:108-122
11. Ruggeri L, Capanni M, Casucci M, et al. Role of natural killer cell alloreactivity in HLA-mismatched hematopoietic stem cell transplantation. Blood. 1999; 94:333-339
12. Farag S S, Fehniger T A, Ruggeri L, Velardi A, Caligiuri M A Natural killer cell receptors: new biology and insights into the graft-versus-leukemia effect. Blood. 2002; 100:1935-1947
13. Velardi A, Ruggeri L, Moretta A, Moretta L NK cells: a lesson from mismatched hematopoietic transplantation. Trends Immunol. 2002; 23:438-444
14. Guven H, Gilijam M, Chambers B J, et al. Expansion of natural killer (NK) and natural killer-like T (NKT)-cell populations derived from patients with B-chronic lymphocytic leukemia (B-CLL): a potential source for cellular immunotherapy. Leukemia. 2003; 17:1973-1980
15. Lauwerys B R, Garot N, Renauld J C, Houssiau F A Cytokine production and killer activity of NK/T-NK cells derived with IL-2, IL-15, or the combination of IL-12 and IL-18. J Immunol. 2000; 165:1847-1853
16. Parrish-Novak J, Dillon S R, Nelson A, et al. Interleukin 21 and its receptor are involved in NK cell expansion and regulation of lymphocyte function. Nature. 2000; 408:57-63
17. Strengell M, Matikainen S, Siren J, et al. IL-21 in synergy with IL-15 or IL-18 enhances IFN-gamma production in human NK and T cells. J Immunol. 2003; 170:5464-5469
18. Liu C C, Perussia B, Young J D The emerging role of IL-15 in NK-cell development. Immunol Today. 2000; 21:113-116
19. Rosenberg S A, Lotze M T, Muul L M, et al. Observations on the systemic administration of autologous lymphokine-activated killer cells and recombinant interleukin-2 to patients with metastatic cancer. N Engl J Med. 1985; 313:1485-1492
20. Stein R C, Malkovska V, Morgan S, et al. The clinical effects of prolonged treatment of patients with advanced cancer with low-dose subcutaneous interleukin-2. Br J Cancer. 1991; 63:275-278
21. Atkins M B, Lotze M T, Dutcher J P, et al. High-dose recombinant interleukin 2 therapy for patients with metastatic melanoma: analysis of 270 patients treated between 1985 and 1993. J Clin Oncol. 1999; 17:2105-2116
22. Chada S, Ramesh R, Mhashilkar A M Cytokine- and chemokine-based gene therapy for cancer. Curr Opin Mol Ther. 2003; 5:463-474
23. Panelli M C, White R, Foster M, et al. Forecasting the cytokine storm following systemic interleukin (IL)-2 administration. J Transl Med. 2004; 2:17
24. Maas R A, Dullens H F, Den Otter W Interleukin-2 in cancer treatment: disappointing or (still) promising? A review. Cancer Immunol Immunother. 1993; 36:141-148
25. Weisdorf D J, Anderson P M, Blazar B R, et al. Interleukin 2 immediately after autologous bone marrow transplantation for acute lymphoblastic leukemia—a phase I study. Transplantation. 1993; 55:61-66
26. Ochoa J B, Curti B, Peitzman A B, et al. Increased circulating nitrogen oxides after human tumor immunotherapy: correlation with toxic hemodynamic changes. J Natl Cancer Inst. 1992; 84:864-867
27. Glauser F L, DeBlois G, Bechard D, et al. Cardiopulmonary toxicity of adoptive immunotherapy. Am J Med Sci. 1988; 296:406-412
28. Ardizzoni A, Bonavia M, Viale M, et al. Biologic and clinical effects of continuous infusion interleukin-2 in patients with non-small cell lung cancer. Cancer. 1994; 73:1353-1360
29. Miller J S, Tessmer-Tuck J, Blake N, et al. Endogenous IL-2 production by natural killer cells maintains cytotoxic and proliferative capacity following retroviral-mediated gene transfer. Exp Hematol. 1997; 25:1140-1148
30. Gong J H, Maki G, Klingemann H G Characterization of a human cell line (NK-92) with phenotypical and functional characteristics of activated natural killer cells. Leukemia. 1994; 8:652-658
31. Nagashima S, Mailliard R, Kashii Y, et al. Stable transduction of the interleukin-2 gene into human natural killer cell lines and their phenotypic and functional characterization in vitro and in vivo. Blood. 1998; 91:3850-3861
32. Tam Y K, Maki G, Miyagawa B, et al. Characterization of genetically altered, interleukin 2-independent natural killer cell lines suitable for adoptive cellular immunotherapy. Hum Gene Ther. 1999; 10:1359-1373
33. Malek T R, Bayer A L Tolerance, not immunity, crucially depends on IL-2. Nat Rev Immunol. 2004; 4:665-674
34. Laker C, Stocking C, Bergholz U, et al. Autocrine stimulation after transfer of the granulocyte/macrophage colony-stimulating factor gene and autonomous growth are distinct but interdependent steps in the oncogenic pathway. Proc Natl Acad Sci U S A. 1987; 84:8458-8462
35. Browder T M, Abrams J S, Wong P M, Nienhuis A W Mechanism of autocrine stimulation in hematopoietic cells producing interleukin-3 after retrovirus-mediated gene transfer. Mol Cell Biol. 1989; 9:204-213

1. A method of immunotherapy using gene modified cells, expressing substantially physiological levels of IL-2, wherein an IL-2 expressing mammalian cell is produced through a method, comprising the steps of
selection or construction of an IL-2 gene,
preparation of a retroviral vector carrying said IL-2 gene,
collection of cells from a donor or patient,
genetic modification of cells,
optionally, selection of gene-modified cells, and
wherein said cells are administered to a patient in need thereof.

2. The method according to claim 1, wherein said IL-2 gene is a gene encoding the protein of SEQ. ID. NO. 11 or functional equivalents thereof.

3. The method according to claim 1, wherein said IL-2 gene is a gene encoding the protein of SEQ. ID. NO. 13 or functional equivalents thereof.

4. The method according to claim 1, wherein said IL-2 gene is a gene chosen among SEQ. ID. NO. 8, SEQ. ID. NO. 10, and functional equivalents thereof.

5. The method according to claim 1, wherein said mammalian cells are chosen among natural killer (NK) cells, and T-cells.

6. The method according to claim 5, wherein said mammalian cells are natural killer (NK) cells.

7. The method according to claim 1, wherein said IL-2 gene is modified to direct the expression of IL-2 to the endoplasmatic reticulum of said cell.

8. A method of producing a gene modified cell expressing substantially physiological levels of IL-2, comprising the steps of
selection or construction of an IL-2 gene,
preparation of a retroviral vector carrying said IL-2 gene,
collection of cells from a donor or patient,
genetic modification of cells,
optionally, selection of the gene modified cells.

9. The method according to claim 8, wherein said IL-2 gene is a gene encoding the protein of SEQ. ID. NO. 11 or functional equivalents thereof.

10. The method according to claim 8, wherein said IL-2 gene is a gene encoding the protein of SEQ. ID. NO. 13 or functional equivalents thereof.

11. The method according to claim 8, wherein said IL-2 gene is a gene chosen among SEQ. ID. NO. 8, SEQ. ID. NO. 10, and functional equivalents thereof.

12. A transgenic mammalian cell capable of producing IL-2, obtainable by the method according to claim 8.

13. A transgenic mammalian cell obtainable by the method according to claim 8, which cell in non-modified state is dependent of IL-2 for its growth, and unable to produce any significant amounts thereof, wherein said transgenic cell produces IL-2 in an amount sufficient to sustain growth without the need of external IL-2.

14. The transgenic mammalian cell according to claim 13, wherein IL-2 is expressed and retained in the endoplasmatic reticulum of said cell.

15. A method in the treatment of cancer, comprising the administration to said patients a therapeutically efficient and physiologically acceptable amount of transgenic cells according to claim 12.

16. The method according to claim 1, wherein said cells are taken from the patient, transfected, and returned to said same patient.

17. The method according to claim 1, wherein said cells are taken from a donor, transfected, and administered to said patient.

18. The method according to claim 1, wherein said use of transgenic cells constitutes an adjunct or supplementary therapy, performed before, after or substantially simultaneously with another therapy.

19. The method according to claim 1, wherein said use of transgenic cells constitutes the primary therapy in the treatment of cancer.

20. The method according to claim 1, wherein said immunostimulation constitutes a step in the treatment or prevention of infection.
